(19) United States
(12) Patent Application Publication
(10) Pub. No.: US 2025/0367479 A1
(43) Pub. Date: Dec. 4, 2025

(54) HIGH-INTENSITY FOCUSED ULTRASONIC WAVE PROVIDING DEVICE CAPABLE OF MOVING IN Y-AXIS DIRECTION

(71) Applicant: SKINGRAB CO., LTD., Seoul (KR)

(72) Inventor: Sugun LEE, Paju-si (KR)

(73) Assignee: SKINGRAB CO., LTD., Seoul (KR)

(21) Appl. No.: 19/219,014

(22) Filed: May 27, 2025

(30) Foreign Application Priority Data
May 28, 2024 (KR) 10-2024-0069159

(51) Int. Cl. A61N 7/02; A61N 7/00

(52) U.S. Cl. CPC A61N 7/02; A61N 2007/0034; A61N 2007/0091

(57) ABSTRACT

Disclosed is a high-intensity focused ultrasonic wave providing device capable of moving in an Y-axis direction. A device, which radiates high-intensity focused ultrasonic waves to a skin, according to the present disclosure includes a handpiece gripped by a hand of a user, a cartridge housing detachably coupled to the handpiece and disposed adjacent to the skin when the ultrasonic waves are radiated to the skin, a transducer that is disposed in the cartridge housing, delivers the ultrasonic waves to the skin, and has a focal length of 0.5 mm to 4.5 mm, a driving device that moves the transducer, and a controller that is electrically connected to the driving device to control the driving device and controls the transducer and the driving device such that an ultrasonic wave radiation pattern having a spiral shape, a zigzag shape, or a wave shape is generated as the transducer moves.

### CROSS-REFERENCE TO RELATED APPLICATIONS

A claim for priority under 35 U.S.C. §119 is made to Korean Patent Application No. 10-2024-0069159 filed on May 28, 2024 in the Korean Intellectual Property Office, the entire contents of which are hereby incorporated by reference.

### BACKGROUND

Embodiments of the present disclosure described herein relate to the field of medical devices for treating a skin and a tissue, and more particularly, relate to a high-intensity focused ultrasound (HIFU) providing device in which a transducer may move in an X-axis direction and a Y-axis direction to deliver precise ultrasonic wave patterns to various skin depths.

An “high intensity focused ultrasound (HIFU)” used herein means ultrasonic waves that have an energy intensity of 0.5 W/cm2 or more and may deliver thermal energy to a depth of 0.5 mm to 4.5 mm in a skin without heat damage to a skin surface while focused at a specific focal length. This HIFU technology induces coagulation necrosis of tissues, resulting in collagen regeneration and elasticity enhancement.

High intensity focused ultrasound (HIFU) devices non-invasively delivers ultrasonic waves to a skin to focus heat energy without causing a damage to a skin surface, and thus induce coagulation necrosis. In this way, a necrotic portion in a deep skin is naturally restored by a repair mechanism of a damaged portion of a human body.

The HIFU device includes a cartridge housing disposed adjacent to the skin and a transducer that is provided in the cartridge housing and delivers the ultrasonic waves to a target depth of the skin.

However, a focused ultrasonic output device according to the related art does not have various patterns for radiating ultrasonic waves and has may not deliver ultrasonic waves at various intensities. Accordingly, it is difficult to implement an optimum ultrasonic wave radiation pattern and intensity according to a purpose of treatment, and thus, there is a limitation in terms of accurately and effectively delivering ultrasonic energy to a target portion.

In detail, when the ultrasonic waves are radiated at a single pattern and intensity, treatment customized to characteristics and conditions of portions of the skin is not possible. Further, simple ultrasonic wave radiation makes it difficult to deliver ultrasonic energy to various layers of a dermis, resulting in limited therapeutic effects. In addition, when the high-intensity ultrasonic waves are radiated more than necessary, a damage to a skin tissue may be caused, and thus there is a risk of side effects.

Thus, it is required to develop a new high-intensity focused ultrasonic device that may diversify ultrasonic wave radiation patterns and intensities according to skin characteristics and treatment purposes and may target various layers in the dermis. Therefore, it is expected that customized skin treatment may be performed and a treatment effect and safety may be increased simultaneously.

### RELATED ART DOCUMENTS

### Patent Documents

Korean Patent Publication No. 10-1772200

### SUMMARY

The present disclosure is intended to solve the following technical problems.

1) Embodiments of the present disclosure provide a high-intensity focused ultrasonic wave providing device capable of implementing various ultrasonic wave radiation patterns, such as a spiral shape, a zigzag shape, and a wave shape, on a skin by providing a transducer capable of precisely moving not only in an X-axis direction but also in an Y-axis direction.
2) Embodiments of the present disclosure also provide a multi-focus transducer system capable of delivering ultrasonic energy optimized for different depths from a surface layer to a deep layer of a skin in a stepwise manner.
3) Embodiments of the present disclosure also provide a control system that allows a user to customize an ultrasonic wave intensity, a pattern, and a treatment depth according to a treatment purpose and characteristics of a skin portion.

According to an embodiment, a device, which radiates high-intensity focused ultrasonic waves to a skin, includes a handpiece gripped by a hand of a user, a cartridge housing detachably coupled to the handpiece and disposed adjacent to the skin when the ultrasonic waves are radiated to the skin, a transducer that is disposed in the cartridge housing, delivers the ultrasonic waves to the skin, and has a focal length of 0.5 mm to 4.5 mm, a driving device that moves the transducer, and a controller that is electrically connected to the driving device to control the driving device and controls the transducer and the driving device such that an ultrasonic wave radiation pattern having a spiral shape, a zigzag shape, or a wave shape is generated as the transducer moves, wherein the driving device includes an X-axis driving device including a linear motor that moves the transducer in an X-axis direction and an Y-axis driving device including a rotation motor that rotates the X-axis driving device to move the transducer in an Y-axis direction, and the X-axis direction and the Y-axis direction are two directions perpendicular to each other in a plane parallel to a skin contact surface of the cartridge housing.

In the embodiment, the X-axis driving device may include the linear motor, and the Y-axis driving device may include the rotation motor.

In the embodiment, the controller may perform a control to synchronize a moving speed and an ultrasonic wave output timing of the transducer and intermittently or continuously output the ultrasonic waves while the transducer moves and adjust a voltage applied to the transducer in a range of 0.5 W/cm² to 100 W/cm² to deliver ultrasonic energy optimized for each skin depth.

In the embodiment, the X-axis driving device and the transducer may be coupled to each other such that the transducer moves in the X-axis direction as the X-axis driving device moves forward or rearward.

In the embodiment, the device may further include a connection part that couples the X-axis driving device and the transducer.

In the embodiment, the controller may rotate the rotation motor in an Y direction and move the linear motor forward or rearward such that the transducer moves in the Y direction after the rotation motor rotates in the Y direction.

In the embodiment, the controller may rotate the rotation motor in an X direction in a state in which the rotation motor rotates in the Y direction and move the linear motor forward or rearward such that the transducer moves in the X direction after the rotation motor rotates in the X direction.

In the embodiment, the rotation motor may be fixed to the handpiece.

In the embodiment, the transducer may include a first transducer disposed in the cartridge housing and a second transducer disposed in the cartridge housing and disposed adjacent to the first transducer, and the first transducer and the second transducer may have different focal lengths and stimulate different depths of the skin through the different focal lengths.

In the embodiment, the driving device may include a first driving device that moves the first transducer in the X-axis direction and the Y-axis direction and a second driving device that moves the second transducer in the X-axis direction and the Y-axis direction, and the first driving device may include a first linear motor and a first rotation motor, and the second driving device may include a second linear motor and a second rotation motor.

In the embodiment, each of the first driving device and the second driving device may include the X-axis driving device that moves the transducer in a first direction and the Y-axis driving device that moves the transducer in a second direction.

In the embodiment, the first transducer may have a focal length for stimulating an epidermis and an upper layer of a dermis of the skin, and the second transducer may have a focal length for stimulating a middle layer and a lower layer of the dermis of the skin.

In the embodiment, the controller may perform a control to radiate the ultrasonic waves from a surface layer to a deep layer of the skin in a stepwise manner by sequentially driving the first transducer and the second transducer.

In the embodiment, the controller may individually control an intensity and an application time of a driving voltage supplied to the first transducer and the second transducer, radiate the ultrasonic waves from a surface layer to a deep layer of the skin in a stepwise manner, and deliver stimulation energy optimized for each skin depth.

According to an embodiment, a method, which radiates high-intensity focused ultrasonic waves to a skin, includes brining a cartridge housing installed in a handpiece into contact with the skin, radiating the ultrasonic waves to a target depth of the skin through a transducer disposed in the cartridge housing, forming a first ultrasonic wave radiation trajectory by moving the transducer in an X-axis direction, changing a movement direction of the transducer to an Y-axis direction through a rotation motor, and forming a second ultrasonic wave radiation trajectory by moving the transducer in the Y-axis direction, wherein a two-dimensional ultrasonic wave radiation pattern is formed by a combination of the first ultrasonic wave radiation trajectory and the second ultrasonic wave radiation trajectory.

### BRIEF DESCRIPTION OF THE FIGURES

The above and other objects and features will become apparent from the following description with reference to the following figures, wherein like reference numerals refer to like parts throughout the various figures unless otherwise specified, and wherein:

FIG. 1 is a perspective view illustrating a high-intensity focused ultrasonic device according to the present disclosure;

FIG. 2 is a perspective view illustrating a state in which the high-intensity focused ultrasonic device according to the present disclosure radiates ultrasonic waves;

FIG. 3 is a perspective view illustrating a driving device according to the present disclosure;

FIG. 4 is a conceptual view illustrating an embodiment in which a transducer is two-dimensionally driven according to the present disclosure;

FIG. 5 is a conceptual view illustrating an embodiment in which a plurality of transducers are included according to the present disclosure;

FIG. 6 is a conceptual view illustrating an embodiment in which the plurality of transducers are driven according to the present disclosure;

FIG. 7 is a conceptual view illustrating the plurality of transducers having different ultrasonic focusing depths;

FIG. 8 is a conceptual view illustrating various patterns which may be radiated by the high-intensity focused ultrasonic device according to the present disclosure; and

FIG. 9 is a flowchart illustrating a method of forming a spiral radiation pattern using the high-intensity focused ultrasonic device according to the present disclosure.

### DETAILED DESCRIPTION

Throughout the present disclosure, the same reference numerals refer to the same components. The present disclosure does not describe all components of embodiments, and general contents or duplicated contents between the embodiments in the technical field to which the present disclosure pertains will be omitted. The terms “unit, module, member, and block” used herein may be implemented in software or hardware, and according to embodiments, the plurality of “units, modules, members, and blocks” may be implemented in one component or one “unit, module, member, and block” may include a plurality of components.

Throughout the specification, when it is described that a first component is “connected” to a second component, this includes not only a case in which the first component is directly connected to the second component but also a case in which the first component is indirectly connected to the second component, and the indirect connection includes connection through a wireless communication network.

Further, when a part “includes” a component, this means that a third component is not excluded but may be further included unless otherwise stated.

Throughout the specification, when a first member is located “on” a second member, this case includes not only a case in which the first member is in contact with the second member but also a case in which a third member is present between the two members.

Terms such as first and second are used to distinguish a first component from a second component, and the components are not limited by the above-described terms.

Singular expressions include plural expressions unless clearly otherwise indicated in the context.

In each operation, an identification code is used for convenience of description and does not describe a sequence of the operations, and an operation may be performed in a different order from a specified order unless the context clearly states a specific order.

Hereinafter, the operating principles and embodiments of the present disclosure will be described with reference to the accompanying drawings.

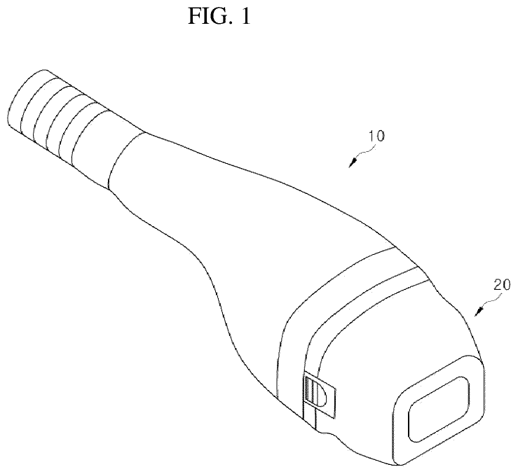

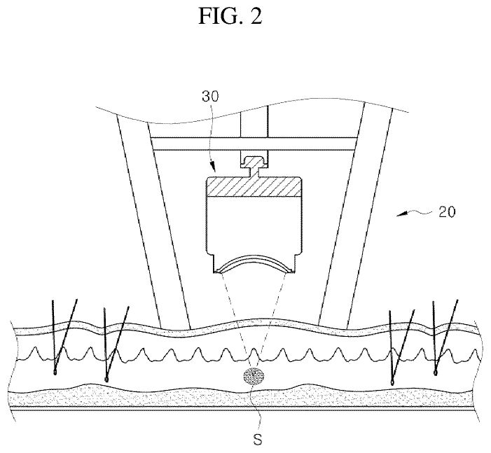

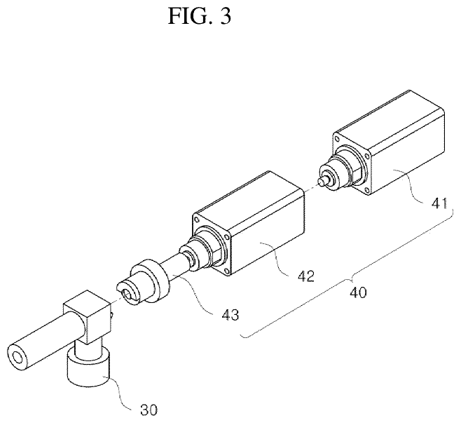

FIG. 1 is a perspective view illustrating a high-intensity focused ultrasonic wave providing device according to an embodiment of the present disclosure, FIG. 2 is a cross-sectional view illustrating an example of a cartridge housing, a transducer, and a controller of the high-intensity focused ultrasonic wave providing device according to the embodiment of the present disclosure, and FIG. 3 is a perspective view illustrating a driving device according to the present disclosure.

Referring to FIGS. 1 to 3, a high-intensity focused ultrasonic wave providing device 100 according to an embodiment disclosed herein includes a handpiece 10, a cartridge housing 20, a transducer 30, a driving device 40, and a controller.

The handpiece 10 is a part gripped by a hand of a user and corresponds to a body on which the cartridge housing 20 is mounted. The cartridge housing 20 is a case detachably coupled to one end of the handpiece 10, and the transducer 30 that generates and delivers ultrasonic waves is installed in the cartridge housing 20. The transducer 30 is a component that is provided as a piezo element or the like and generates ultrasonic waves under control of the controller. The driving device 40 includes an actuator or motor that mechanically controls a position of the transducer 30, and the controller is a microprocessor-based control module that comprehensively controls operations of the transducer 30 and the driving device 40.

Meanwhile, as illustrated in FIG. 3, the transducer 30 may be moved by the driving device 40 in an X-axis direction and a Y-axis direction, and therefore, may form ultrasonic wave radiation trajectories having various patterns on a skin surface.

The handpiece 10 is a basic body and may be used as a handle gripped by the user.

The cartridge housing 20 is detachably coupled to one side of the handpiece 10.

The handpiece 10 may move along a target depth of a skin in a state in which the cartridge housing 20 is in contact with the target depth of the skin. In this case, the handpiece 10 may be manually moved by the user gripping the handpiece 10.

The cartridge housing 20 may be coupled to one side of the handpiece 10 and may be disposed adjacent to the skin. The cartridge housing 20 is a type of case accommodating the transducer 30.

A fluid medium for delivering ultrasonic waves generated by the transducer 30 may be accommodated in the cartridge housing 20.

Water, physiological saline, gel, oil, and the like may be used as the fluid medium, and preferably, a bio gel having no skin irritation and excellent ultrasonic wave transmission efficiency may be applied. This bio gel may be made of a hydrophilic polymer material to maximize a contact area with the skin, to minimize attenuation of ultrasonic waves, and to increase energy transfer efficiency.

Further, the fluid medium may contain various cosmetic ingredients or drugs that help calm and regenerate the skin. Therefore, an additional effect of alleviating side effects of skin procedures and promoting recovery may be expected.

Meanwhile, to adjust a temperature of the fluid medium, a temperature adjusting means such as a heating element or a Peltier element may be provided in the cartridge housing 20. This may warm a procedure site, promote blood flow, and help activate skin metabolism.

The fluid medium may be provided integrally with the cartridge housing 20 or may be provided in a replaceable form through a separate inlet. It is preferable that the fluid medium be used for single use for hygienic use, and it is important to prevent degradation by maintaining a sealed state before and after use.

The handpiece 10 and the cartridge housing 20 of the present disclosure are designed to maximize convenience of an operator and skin contact stability through ergonomic design.

First, the handpiece 10 may be designed to have a shape and size optimized for a hand of the operator, and thus hand fatigue may be minimized even when the handpiece 10 is used for a long time. An uneven pattern or a silicone grip sense for preventing slipping is applied to a surface of the handpiece 10 to provide a stable grip sense. Further, an ultrasonic wave output button is disposed at an upper end of the handpiece 10 at a position that may be operated using a thumb, thereby ensuring convenient operability.

Meanwhile, the cartridge housing 20 may increase an area of a skin contact surface to maintain stable contact. To this end, a curved shape is applied to the skin contact surface of the cartridge housing 20, and an elastic packing is provided at an edge to increase adhesion to the skin. This may increase ultrasonic wave transmission efficiency and prevent skin discomfort due to excessive contact pressure.

Further, a medical polymer material having no skin irritation and excellent durability is applied as materials of the handpiece 10 and the cartridge housing 20. This is a material that is harmless to the human body while satisfying both lightweight and durability and is widely used in medical devices. Accordingly, a high-intensity focused ultrasonic device according to the present disclosure may secure both safety and durability.

Furthermore, the handpiece 10 and the cartridge housing 20 are designed in a separate structure, and thus the cartridge housing 20 may be used as a disposable consumable. This maximizes hygiene of the skin contact surface simultaneously while preventing a risk of cross-infection between users. The fluid medium formed integrally with the cartridge housing 20 is also designed to be easily replaced, thereby enabling customized replacement for each user.

These ergonomic design and material selection are important factors directly related to actual utilization of the device of the present disclosure, and may greatly contribute to enhancing convenience and safety of the user and further product competitiveness.

The transducer 30 may be provided in the cartridge housing 20 to deliver ultrasonic waves to the target depth of the skin. In detail, the transducer 30 may output a frequency of 1 MHz to 10 MHz and generate ultrasonic waves at energy intensities of 0.5 W/cm² to 25 W/cm². The transducer 30 is generally designed to have a diameter of 6.5 mm to 25 mm and to provide a focal length of 0.5 mm to 4.5 mm depth. The transducer 30 is provided to be movable in the cartridge housing 20 in a vertical direction or a horizontal direction and is precisely controlled in the X-axis direction and the Y-axis direction by the driving device 40. In this case, the transducer 30 may be moved by the driving device 40 with a position accuracy of 0.1 mm, and a moving speed may be adjusted in a range of 0.5 mm/s to 5 mm/s. The driving device 40 may be implemented as a linear motor, a step motor, a servo motor, or various types of actuators.

Meanwhile, the cartridge housing 20 may be provided with an electrical energy output unit that outputs ultrasonic waves, light emitting diode (LED) light, light, lasers, high-frequency waves, magnetic fields, or electric fields. Thus, when the transducer 30 outputs ultrasonic waves, the electrical energy output unit may output ultrasonic waves, LED light, light, lasers, high-frequency waves, magnetic fields, or electric fields. That is, the electrical energy output unit simultaneously output ultrasonic waves, LED light, light, lasers, high-frequency waves, magnetic fields, or electric fields together with ultrasonic waves.

The electrical energy output unit is disposed inside the cartridge housing 20 and is configured to output, to the skin, various types of electrical energy such as ultrasonic waves, LED light, light, lasers, high-frequency waves, magnetic fields, and electric fields.

The electrical energy output unit may include a plurality of sub-units, and the sub-units may be divided into an ultrasonic wave output unit, an LED light output unit, a light output unit, a laser output unit, a high frequency output unit, a magnetic field output unit, and an electric field output unit according to an energy source.

The ultrasonic wave output unit is operated together with the transducer 30, and the LED light output unit includes a plurality of LED elements for irradiating the skin with LED rays having various wavelength bands. The light output unit and the laser output unit may be provided as a light source and an optical system for delivering light energy to the skin.

The high frequency output unit includes a radio frequency (RF) energy source that generates high-frequency waves and an electrode that delivers the high-frequency waves to the skin. The magnetic field output unit includes a magnetic field generating coil for applying a magnetic field having a specific intensity to the skin, and the electric field output unit includes an electric field generating electrode for allowing a microcurrent to flow to the skin.

Each unit of the electrical energy output unit may be operated alone or in combination according to a control signal of the controller. In particular, it is known that when ultrasonic waves, LED light, lasers, and the like are radiated together, there is a synergy effect in collagen production and elasticity increase in the skin. Further, when ultrasonic waves, high-frequency waves, electric fields, and the like are used together, energy may be delivered into a deep skin, which may be more effective in improving wrinkles.

The driving device 40 serves to control an ultrasonic wave radiation position by moving the transducer 30 in the X-axis direction and the Y-axis direction. The driving device 40 may be implemented in various forms including the driving motor, the actuator, a link mechanism, and the like.

As the embodiment, referring to FIG. 3, the driving device 40 includes a precision mechanism that enables two-dimensional movement of the transducer 30. In detail, the driving device 40 includes a linear motor 42 for driving in the X-axis direction, a connection part 43 connecting the linear motor 42 and the transducer 30, and a rotation motor 41 fastened to the linear motor 42 to rotate all the linear motor 42, the connection part 43, and the transducer 30 in the Y-axis direction. A principle of operation of this mechanism will be described below. The rotation motor 41 is fixed to the housing and rotates about a rotary shaft together with the linear motor 42. When the rotation motor 41 is rotated, a movement direction of the linear motor 42 is changed according to the rotation. For example, when the rotation motor 41 is rotated by 90 degrees, a driving direction of the linear motor 42 is changed from an X axis to an Y axis. The connection part 43 is a component physically connecting the linear motor 42 and the transducer 30 and is mainly made of a lightweight metal or a reinforced polymer to enable precise force transmission. This connection part 43 transmits a linear motion of the linear motor 42 to the transducer 30 so that the transducer 30 may be moved accurately in both the X axis and the Y axis.

In detail, the controller, which will be described later, may control the linear motor 42 and the rotation motor 41 to drive the transducer 30 in the X-axis direction and the Y-axis direction. The linear motor 42 is coupled to the transducer 30 through the connection part 43, and the transducer 30 is moved in the X-axis direction through forward and backward movement of the linear motor 42. Meanwhile, the rotation motor 41 is fixed to the handpiece 10, and a rotation part in which rotational motion is generated as the rotation motor 41 is driven is coupled to the linear motor 42. Accordingly, when the rotation motor 41 is rotated, the linear motor 42, the connection part 43, and the transducer 30 are rotated together in a rotation direction. Accordingly, a forward and rearward direction of the linear motor 42 coincides with the Y-axis direction, and as the linear motor 42 is moved forward and rearward, the transducer 30 is moved in the Y-axis direction.

When the rotation motor 41 is rotated in a direction opposite to the previously rotated direction, the linear motor 42, the connection part 43, and the transducer 30 are rotated together. Accordingly, the forward and rearward direction of the linear motor 42 coincides with the X-axis direction, and as the linear motor 42 is moved forward and rearward, the transducer 30 is moved in the X-axis direction.

As described above, a high-intensity focused ultrasonic wave radiation device according to the present disclosure may move the transducer 30 in the X-axis direction and the Y-axis direction through interlocking between the linear motor 42 and the rotation motor 41.

Meanwhile, the cartridge housing 20 is filled with degassing water and is designed such that the degassing water does not escape to the outside according to the movement of the transducer 30.

As another embodiment, the driving device 40 may include a first stepping motor for driving in the X-axis direction and a second stepping motor for driving in the Y-axis direction. A timing belt is connected to a rotary shaft of the stepping motor, and the timing belt is connected to a moving block on which the transducer 30 is mounted. Thus, as the stepping motor rotates forward and backward according to the control signal of the controller, the transducer 30 moves in a front-rear direction and a left-right direction.

As another embodiment, the first linear actuator and the second linear actuator may be employed as driving sources in the X-axis direction and the Y-axis direction. In this case, linear motion of the actuator is directly transmitted to the transducer 30, and thus power transmission efficiency may be increased, and position control accuracy may be improved. Further, a precision transfer mechanism such as a ball screw may be applied to enable micrometer positioning.

Meanwhile, a height adjusting mechanism may be provided in the cartridge housing 20 to adjust a position of the transducer 30 in a Z-axis direction. This is to control a skin penetration depth of ultrasonic waves by actively adjusting a distance from the skin surface. The height adjusting mechanism may be implemented as a Z-axis actuator mounted vertically on a base portion of the transducer 30 or by a jack screw method of adjusting a height of the cartridge housing 20.

The components of the driving device 40 cooperate under integrated control of the controller. The controller calculates target positions and moving speeds in the X-axis direction, the Y-axis direction, and the Z-axis direction, generates control signals corresponding thereto, and transmits the control signals to the driving sources. In this case, a position sensor such as an encoder or an optical scale may be additionally provided for real-time position sensing of the transducer 30, and positioning accuracy may be increased through feedback control.

The controller may provide a composite energy output mode according to a user setting, and thus may select an optimum energy combination suitable for a skin condition and a treatment purpose. Therefore, a customized skin remodeling device that meets various dermatological treatment purposes may be implemented.

Further, in the transducer 30, a time during which ultrasonic waves are output may be adjusted by a timer. Here, in the timer, the time may be set by the user. Thus, the user may set a time during which the transducer 30 outputs ultrasonic waves using the timer, and thus adjust a time during which a procedure of applying the ultrasonic waves output from the transducer 30 to the target depth of the skin is performed.

The transducer 30 may receive an electric signal from a power source of the handpiece 10 and focus ultrasonic waves to a specific position, and the ultrasonic waves may be referred to as high-intensity focused ultrasonic waves.

The controller may be disposed on the handpiece 10 or the cartridge housing 20 to control the transducer 30 and the driving device 40. The controller may control the driving device 40 so that the transducer 30 moves in at least one of a first direction parallel to the skin and a second direction perpendicular to the first direction and parallel to the skin.

Here, the first direction and the second direction may be set based on the skin in contact with the cartridge housing 20 and may be set based on one surface of the cartridge housing 20 in contact with the skin. That is, the first direction and the second direction may be a direction parallel to the skin in a state in which the cartridge housing 20 is in contact with the skin or a direction parallel to one surface of the cartridge housing 20 in contact with the skin.

The controller may control the transducer 30 such that the transducer 30 radiates ultrasonic waves toward the skin while moving in any one of the first direction and the second direction.

In the specification, to describe the movement of the transducer 30, reference numerals of the transducer before the movement are indicated by 30, 30a, and 30b, and reference numerals of the transducer after the movement are indicated by 30′, 30a′, and 30b′.

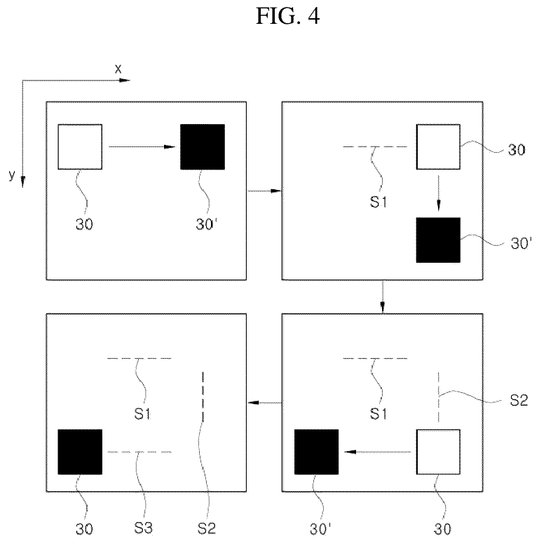

For example, referring to FIG. 4, the controller moves the transducer 30 in the first direction. In this case, the controller allows the transducer 30 to radiate ultrasonic waves. Accordingly, a line-shaped ultrasonic wave radiation trajectory S1 may be formed on the skin. Meanwhile, the controller controls the transducer 30 such that the transducer 30 radiates ultrasonic waves while moving in the second direction. Accordingly, a ultrasonic wave radiation trajectory S2 formed in the second direction is formed. In the same manner, the controller may allow an ultrasonic wave radiation trajectory S3 in the first direction to be formed on the skin. As a result, the device according to the present disclosure may radiate ultrasonic waves while drawing a two-dimensional pattern.

To this end, the driving device 40 may include an X-axis driving device for moving the transducer 30 in the first direction and a Y-axis driving device for moving the transducer 30 in the second direction.

In the embodiment, each of the X-axis driving device and the Y-axis driving device may be fixed to any one of the handpiece 10 and the cartridge housing 20 to move the transducer 30 with respect to the corresponding fixed position.

In the embodiment, the X-axis driving device may be fixed to the cartridge housing 20, and the Y-axis driving device may be fixed to the handpiece 10.

In the embodiment, the X-axis driving device may be fixed to the handpiece 10, and the Y-axis driving device may be fixed to the cartridge housing 20.

Meanwhile, the high-intensity focused ultrasonic wave providing device according to the present disclosure may include a plurality of transducers.

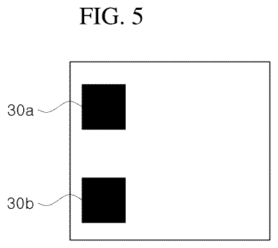

In the embodiment, referring to FIG. 5, the transducer 30 may include a first transducer 30a disposed in the cartridge housing 20 and a second transducer 30b disposed in the cartridge housing 20 and disposed adjacent to the first transducer 30a.

In the embodiment, the driving device 40 may include a first driving device for moving the first transducer 30a in at least one of the first direction and the second direction and a second driving device for moving the second transducer 30b in at least one of the first direction and the second direction.

In the embodiment, each of the first driving device and the second driving device may include an X-axis driving device for moving the transducer 30 in the first direction and a Y-axis driving device for moving the transducer 30 in the second direction.

That is, the first transducer 30a and the second transducer 30b may move in the X-axis direction and the Y-axis direction.

In the embodiment, the controller may control the first transducer 30a and the first driving device 40 such that the first transducer 30a radiates ultrasonic waves to the skin while moving in the first direction, may control the first driving device 40 such that the first transducer 30a moves in the second direction, and may control the second transducer 30b and the second driving device 40 such that the second transducer 30b radiates ultrasonic waves to the skin while moving along a trajectory along which the first transducer 30a radiates ultrasonic waves.

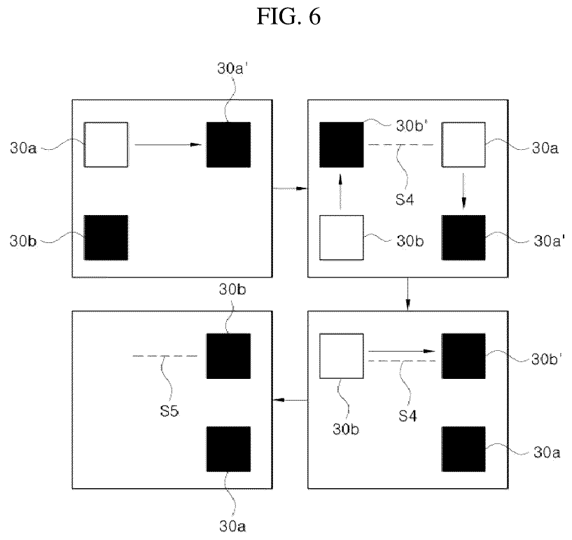

Referring to FIG. 6, the controller controls the first transducer 30asuch that the first transducer 30a radiates ultrasonic waves to the skin while moving in the first direction. Accordingly, an ultrasonic wave radiation trajectory S4 in the first direction is formed. Thereafter, the controller moves the first transducer 30a in the second direction and moves the second transducer 30b in an opposite direction to the second direction. Thereafter, the controller controls the second transducer 30b and the second driving device 40 so that the second transducer 30b radiates ultrasonic waves to the skin while moving along the trajectory S4 along which the first transducer 30a radiates ultrasonic waves. Accordingly, a trajectory S5 obtained by radiating ultrasonic waves twice at the same position is formed.

As illustrated in FIGS. 5 and 6, the plurality of transducers including the first transducer 30a and the second transducer 30b may be provided. In this case, the transducers 30a and 30b may be independently controlled by the corresponding driving devices 40, 40a, and 40b, and thus ultrasonic waves may be radiated to the skin in an overlapping manner (S5).

In the embodiment, the controller may control the first transducer 30a and the second transducer 30b so that the first transducer 30a and the second transducer 30b radiate ultrasonic waves having different energies to the skin.

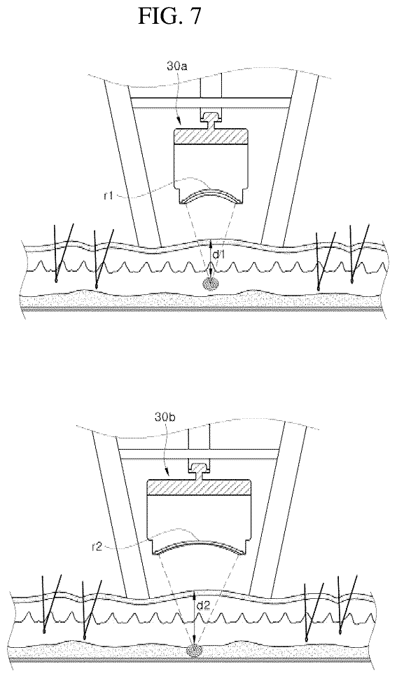

In the embodiment, the first transducer 30a and the second transducer 30b may be formed to radiate ultrasonic waves to the skin at different depths. Referring to FIG. 7, the first transducer 30a and the second transducer 30b include focusing parts r1 and r2 having different radii of curvature. Therefore, the first transducer 30a and the second transducer 30b have different focusing depths d1 and d2.

According to the embodiment, the plurality of transducers 30a and 30b having different focal lengths may be provided in the cartridge housing 20. In this case, the controller may individually control ultrasonic wave output timings of the transducers, and thus implement a stepwise stimulation effect according to a depth of the skin.

In detail, the first transducer 30a close to the skin may be first driven to apply ultrasonic stimulation to an epidermis portion, and next, the second transducer 30b capable of delivering ultrasonic waves to the deep skin may be driven to stimulate a dermis portion. A skin tissue is deepened in an order of epidermis-dermis-subcutaneous fat, and thus stepwise and systematic stimulation may be applied to the entire layers of the skin through the sequential ultrasonic wave radiation.

Furthermore, the controller may optimize the amount of stimulation at each skin depth by adjusting driving times and output intensities of the transducers 30a and 30b. For example, a customized procedure effect may be implemented in a manner of applying stronger and longer stimulation to the dermis portion at which collagen is actively produced and minimizing stimulation to the epidermis portion.

Further, an optimum stimulation pattern according to the procedure site such as a face, a neck, and an arm may be applied in consideration of differences between skin thicknesses at the portions. To this end, a procedure condition for each skin portion may be stored in the form of a database in the controller, and a control protocol suitable for the corresponding portion may be automatically selected and executed according to an input of the user.

In this way, by applying differentiated ultrasonic stimulation to various skin depths through the plurality of transducers, it is possible to induce a biological reaction required for skin regeneration in a stepwise manner and maximize the synergy effect. This may be a unique feature and advantage of the present disclosure, which is difficult to achieve with conventional single-transducer methods.

In the embodiment, the controller may control the first driving device and the second driving device so that moving speeds of the first transducer 30a and the second transducer 30b are different from each other.

The controller may cooperatively control the ultrasonic wave output timing of the transducer 30 and the operation of the driving device 40 and thus radiate ultrasonic waves to the skin with various trajectories, patterns, energy intensities, and speeds.

In detail, the controller moves the transducer 30 on an X-Y plane through the driving device 40, turns on/off ultrasonic wave oscillation of the transducer 30 in synchronization with this movement, and thus implements an ultrasonic wave radiation trajectory having a desired shape. For example, when the transducer 30 intermittently outputs ultrasonic waves while moving in the X-axis direction, a dotted line-shaped radiation trajectory may be obtained, and when the transducer 30 continuously outputs ultrasonic waves while alternately moving in the X-axis direction and the Y-axis direction, a zigzag-shaped radiation trajectory may be obtained.

Furthermore, the controller may adjust an intensity of ultrasonic energy delivered to the skin tissue by variably controlling an applied voltage or oscillation frequency to the transducer 30. Therefore, the energy may be delivered from a surface layer to a deep layer of the skin, thereby enhancing a skin regeneration effect.

Further, the controller may set the moving speed or ultrasonic wave output timing of the transducer 30, and thus adjust an amount of radiated ultrasonic energy per unit area.

Meanwhile, when the plurality of transducers 30a and 30b are provided, the controller may individually control the ultrasonic wave output timings of the transducers and assign a relative phase difference or time difference therebetween. Therefore, a plurality of ultrasonic beams may focus energy while causing interference or resonance in a specific area of the skin.

Finally, the controller may optimize parameters such as the ultrasonic wave radiation trajectory/pattern/energy/speed in real time based on skin characteristic information or treatment mode selection information input through a user interface unit. Accordingly, the user may conveniently set and apply a high-intensity ultrasonic wave radiation protocol customized for a skin type or a treatment purpose.

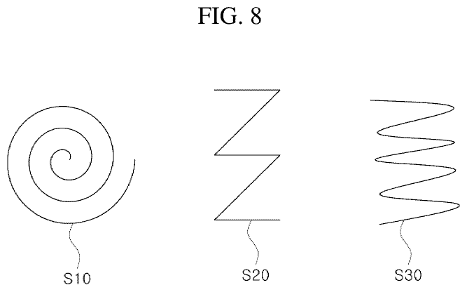

Meanwhile, the controller may implement various ultrasonic wave radiation patterns by controlling a two-dimensional behavior of the transducer 30. Examples of representative ultrasonic wave radiation patterns include a spiral shape, a zigzag shape, and a wave shape, as illustrated in FIG. 8.

A spiral pattern S10 is formed by a method in which the transducer 30 radiates ultrasonic waves while moving along a spiral trajectory, and a radiation area is expanded from a central portion to the outside. This is effective when a wide portion of the skin is stimulated without omission.

A zigzag pattern S20 is formed by a method in which the transducer 30 radiates ultrasonic waves along a zigzag trajectory, and reciprocating movements in the X-axis direction and the Y-axis direction are alternately performed. This is suitable for intensively stimulating a relatively narrow area.

Meanwhile, a wave pattern S30 is formed by a method of radiating ultrasonic waves along a gentle curve like a sine wave. This may be effectively used to improve wrinkles by naturally radiating ultrasonic waves along long straight wrinkles.

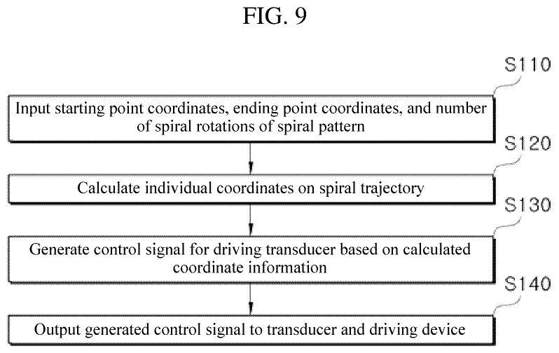

The ultrasonic wave radiation patterns are implemented by a control algorithm executed by the controller. FIG. 9 is a flowchart illustrating, by way of example, an operation sequence of the controller for implementing the spiral pattern S10.

First, when parameters such as starting point coordinates (X0, Y0), ending point coordinates (X1, Y1), and the number (N) of spiral rotations of the spiral pattern S10 are input from the user (S100), the controller calculates individual coordinates (Xi, Xi) on the spiral trajectory (S110). Further, based on the calculated coordinate information, a control signal for driving the transducer 30 is generated (S120) and is output to the driving device 40 (S130). In this case, the ultrasonic wave output timing of the transducer 30 is together controlled (S140), and thus the intended spiral pattern S10 is formed on the skin surface.

In the case of other patterns such as a zigzag shape or a wave shape, the controller may calculate and output a driving signal of the transducer 30 and thus obtain a desired radiation pattern in the same manner. In this case, a shape, a size, and a density of the pattern may be selectively input through an user interface, and thus customized ultrasonic wave radiation according to the skin condition and a procedure purpose may be performed.

This two-dimensional ultrasonic wave scanning technology may significantly improve a skin irritation effect as compared to conventional simple reciprocating ultrasonic wave radiation. In addition, predictability and reproducibility of the procedure may be increased through use of various radiation patterns, thereby enabling stable procedure quality implementation.

In controlling ultrasonic wave output of the transducer 30, the controller of the present disclosure may provide an ultrasonic wave radiation mode customized and optimized for the skin type and disease characteristics of the user. To this end, the user interface may be provided in the handpiece 10, a separate touch screen, or the like.

The user interface may receive basic information such as the skin type (a dry type, a neutral type, and an oily type), an age, and a gender to identify skin characteristics of the user, and recommend a suitable ultrasonic wave radiation mode based on these skin characteristics. For example, in the case of a dry skin having advanced aging, a high-intensity mode that may deliver ultrasonic waves to a deeper dermis layer may be suggested, and in the case of a sensitive skin, a low-intensity mode having less irritation may be suggested.

Further, when the user selects a skin disease or a problem (wrinkles, pigmentation, acne, elasticity loss, and the like) to be improved, the ultrasonic wave radiation pattern and intensity that are effective for the disease may be customized. In this case, an optimum procedure condition preset for each disease is loaded from the database and applied, and thus the user may simply use the customized procedure mode without cumbersomely inputting detailed conditions.

Furthermore, a more intuitive and convenient interface may be provided by graphically displaying the procedure site (e.g., a forehead, eyes, cheeks, a neck, or the like) and allowing the user to perform a touch input. Therefore, a procedure mode specialized in the selected portion is automatically set and is thus used to drive the transducer 30.

This user-customized interface may contribute to increasing utilization and marketability of the device by allowing general users not professional practitioners to easily use the device. Further, a skin improvement effect may be maximized through the procedure mode optimized for each skin type and each disease, which is an important technical feature that may further increase usefulness of the present disclosure.

The high-intensity focused ultrasonic device according to the present disclosure may increase a treatment effect by precisely radiating ultrasonic waves to the skin in various two-dimensional patterns. The controller may freely set the ultrasonic wave radiation trajectory of the transducer 30 on an X-Y plane, thereby enabling customized ultrasonic wave radiation suitable for characteristics of each portion of the skin.

Furthermore, the present disclosure may variably adjust an intensity of ultrasonic waves through driving of the transducer 30. Therefore, ultrasonic waves having a suitable intensity may be delivered to various depths from an epidermal layer to a dermal layer of the skin, and thus a uniform and effective collagen regeneration effect throughout the skin may be expected.

Further, according to the present disclosure, ultrasonic waves may be simultaneously radiated to a wide range of the skin using the plurality of transducers. In this case, the controller may control the ultrasonic beams output from the plurality of transducers such that the ultrasonic beams overlap each other at the same point on the skin, thereby focusing high-intensity ultrasonic energy on the corresponding portion. This is expected to be usefully used to increase a tightening or lifting effect at a local portion of the skin.

In addition, in the present disclosure, sliding movement and the ultrasonic wave output timing of the transducer may be controlled in a programming manner. Accordingly, it is possible to implement an optimum ultrasonic wave pattern according to a treatment portion and purpose, such as a two-dimensional ultrasonic wave scanning mode and a wave-type and zigzag-type radiation mode.

In this way, the present disclosure may achieve a dramatically improved skin treatment effect compared to the conventional HIFU technology through two-dimensional behavior control of high-intensity focused ultrasonic waves.

The present disclosure provides various important technical improvements compared to conventional high-intensity focused ultrasonic devices. In particular, Korean Patent Publication No. 10-1772200 discloses a transducer that may move only in one direction and thus has a limitation in that only simple linear-pattern ultrasonic wave radiation is possible.

On the other hand, in the present disclosure, 1) through an unique combination of the rotation motor and the linear motor, the two-dimensional movement in the X-axis direction and the Y-axis direction may be performed, and various ultrasonic wave radiation patterns such as a spiral shape, a zigzag shape, and a wave shape may be implemented.

2) By using the plurality of transducers having different focal lengths, ultrasonic waves may be radiated from the surface layer to the deep layer of the skin a stepwise manner, and thus stimulation energy optimized for each skin depth may be delivered.

3) By complexly controlling the moving speed and the ultrasonic wave output timing of the transducer, the amount of radiated ultrasonic energy per unit area may be precisely adjusted.

Through these technical improvements, the present disclosure may provide a skin elasticity improvement effect increased by about 30% to 50% and a procedure time reduced by about 40% compared to the related art.

It is considered that the present disclosure may be widely used in treatment of various skin diseases such as freckles, moles, blemishes, acne, and scars and in a skin care field.

A high-intensity focused ultrasonic wave providing device according to the present disclosure includes a transducer that may move on a two-dimensional plane. Therefore, the present disclosure may radiate ultrasonic waves in various patterns.

Further, the high-intensity focused ultrasonic wave providing device according to the present disclosure may include the plurality of transducers, and thus radiate, to the skin, ultrasonic waves having various depths, various patterns, and various energies. Therefore, the present disclosure may improve a treatment effect through ultrasonic wave radiation.

The effects of the present disclosure are not limited to the effects described above, and those skilled in the art will clearly understand other effects not described based on the following description.

Embodiments disclosed with reference to the accompanying drawings have been described as above. Those skilled in the art to which the present disclosure pertains may understand that the present disclosure may be implemented in forms different from those of the disclosed embodiments without changing the technical spirit or essential features of the present disclosure. The disclosed embodiments are illustrative and should not be construed restrictively.

What is claimed is:

1. A high-intensity focused ultrasonic wave providing device, which radiates high-intensity focused ultrasonic waves to a skin, the device comprising:
a handpiece gripped by a hand of a user;
a cartridge housing detachably coupled to the handpiece and disposed adjacent to the skin when the ultrasonic waves are radiated to the skin;
a transducer disposed in the cartridge housing, configured to deliver the ultrasonic waves to the skin, and having a focal length of 0.5 mm to 4.5 mm;
a driving device configured to move the transducer; and
a controller electrically connected to the driving device to control the driving device and configured to control the transducer and the driving device such that an ultrasonic wave radiation pattern having a spiral shape, a zigzag shape, or a wave shape is generated as the transducer moves,
wherein the driving device includes:
an X-axis driving device including a linear motor configured to move the transducer in an X-axis direction; and
an Y-axis driving device including a rotation motor configured to rotate the X-axis driving device to move the transducer in an Y-axis direction, and
wherein the X-axis direction and the Y-axis direction are two directions perpendicular to each other in a plane parallel to a skin contact surface of the cartridge housing.

2. The high-intensity focused ultrasonic wave providing device of claim 1, wherein the X-axis driving device includes the linear motor, and
wherein the Y-axis driving device includes the rotation motor.

3. The high-intensity focused ultrasonic wave providing device of claim 1, wherein the controller is configured to:
perform a control to synchronize a moving speed and an ultrasonic wave output timing of the transducer and intermittently or continuously output the ultrasonic waves while the transducer moves; and
adjust a voltage applied to the transducer in a range of 0.5 W/cm² to 100 W/cm² to deliver ultrasonic energy optimized for each skin depth.

4. The high-intensity focused ultrasonic wave providing device of claim 2, wherein the X-axis driving device and the transducer are coupled to each other such that the transducer moves in the X-axis direction as the X-axis driving device moves forward or rearward.

5. The high-intensity focused ultrasonic wave providing device of claim 4, further comprising:
a connection part configured to couple the X-axis driving device and the transducer.

6. The high-intensity focused ultrasonic wave providing device of claim 5, wherein the controller is configured to:
rotate the rotation motor in an Y direction; and
move the linear motor forward or rearward such that the transducer moves in the Y direction after the rotation motor rotates in the Y direction.

7. The high-intensity focused ultrasonic wave providing device of claim 6, wherein the controller is configured to:
rotate the rotation motor in an X direction in a state in which the rotation motor rotates in the Y direction; and
move the linear motor forward or rearward such that the transducer moves in the X direction after the rotation motor rotates in the X direction.

8. The high-intensity focused ultrasonic wave providing device of claim 7, wherein the rotation motor is fixed to the handpiece.

9. The high-intensity focused ultrasonic wave providing device of claim 1, wherein the transducer includes:
a first transducer disposed in the cartridge housing; and
a second transducer disposed in the cartridge housing and disposed adjacent to the first transducer, and
wherein the first transducer and the second transducer have different focal lengths and stimulate different depths of the skin through the different focal lengths.

10. The high-intensity focused ultrasonic wave providing device of claim 9, wherein the driving device includes:
a first driving device configured to move the first transducer in the X-axis direction and the Y-axis direction; and
a second driving device configured to move the second transducer in the X-axis direction and the Y-axis direction, and
wherein the first driving device includes a first linear motor and a first rotation motor, and the second driving device includes a second linear motor and a second rotation motor.

11. The high-intensity focused ultrasonic wave providing device of claim 10, wherein each of the first driving device and the second driving device includes:
the X-axis driving device configured to move the transducer in a first direction; and
the Y-axis driving device configured to move the transducer in a second direction.

12. The high-intensity focused ultrasonic wave providing device of claim 9, wherein the first transducer has a focal length for stimulating an epidermis and an upper layer of a dermis of the skin, and
wherein the second transducer has a focal length for stimulating a middle layer and a lower layer of the dermis of the skin.

13. The high-intensity focused ultrasonic wave providing device of claim 9, wherein the controller performs a control to radiate the ultrasonic waves from a surface layer to a deep layer of the skin in a stepwise manner by sequentially driving the first transducer and the second transducer.

14. The high-intensity focused ultrasonic wave providing device of claim 9, wherein the controller individually controls an intensity and an application time of a driving voltage supplied to the first transducer and the second transducer, radiates the ultrasonic waves from a surface layer to a deep layer of the skin in a stepwise manner, and delivers stimulation energy optimized for each skin depth.

15. A high-intensity focused ultrasonic wave radiating method, which radiates high-intensity focused ultrasonic waves to a skin, the method comprising:
brining a cartridge housing installed in a handpiece into contact with the skin;
radiating the ultrasonic waves to a target depth of the skin through a transducer disposed in the cartridge housing;
forming a first ultrasonic wave radiation trajectory by moving the transducer in an X-axis direction;
changing a movement direction of the transducer to an Y-axis direction through a rotation motor; and
forming a second ultrasonic wave radiation trajectory by moving the transducer in the Y-axis direction,
wherein a two-dimensional ultrasonic wave radiation pattern is formed by a combination of the first ultrasonic wave radiation trajectory and the second ultrasonic wave radiation trajectory.
